(19) United States
(12) Patent Application Publication
(10) Pub. No.: US 2022/0208299 A1
(43) Pub. Date: Jun. 30, 2022

(54) SYSTEMS AND METHODS FOR LABORATORY STATISTICAL ANALYSIS

(71) Applicant: Future Technologies, Inc., Fairfax, VA (US)

(72) Inventor: Ram N. Gupta, Fairfax, VA (US)

(21) Appl. No.: 17/555,185

(22) Filed: Dec. 17, 2021

Related U.S. Application Data
(60) Provisional application No. 63/132,247, filed on Dec. 30, 2020.

(51) Int. Cl. G16B 10/00; G16B 45/00; G16B 40/20

(52) U.S. Cl. CPC G16B 10/00; G16B 40/20; G16B 45/00

(57) ABSTRACT

An aspect of the disclosed embodiments includes a system for analysis. The system includes a processor and a memory. The memory includes instructions that, when executed by the processor, cause the processor to receive first initial DNA profile data from a first computing device, receive second initial DNA profile data from a second computing device and compile the first initial DNA profile data and second initial DNA profile data into a first combined DNA profile data file.

### CROSS-REFERENCES TO RELATED APPLICATIONS

This patent application claims priority to U.S. Provisional Patent Application Ser. No. 63/132,247, filed Dec. 30, 2020 which is incorporated herein by reference in its entirety.

### FIELD

This disclosure generally relates to a computer systems and methods for laboratory statistical analysis.

### BACKGROUND

Various laboratory software is used to analyze and present information relating to deoxyribonucleic acid (DNA) identification. In some cases, DNA profiles of individuals from different sources may differ and need to be reconciled. Further, in some cases, this DNA information may be useful in determining family trees.

### SUMMARY

This disclosure generally relates to laboratory statistical analysis.

An aspect of the disclosed embodiments includes a system for analysis. The system includes a processor and a memory. The memory includes instructions that, when executed by the processor, cause the processor to receive first initial DNA profile data from the first computing device, receive a second initial DNA profile data from a second computing device and compile the first initial DNA profile data and second initial DNA profile data into a first combined DNA profile data file.

Another aspect of the disclosed embodiments includes a method for analysis. The method includes receiving first initial DNA profile data from a first computing device, receiving second initial DNA profile data from a second computing device, and compiling the first initial DNA profile data and second initial DNA profile data into a first combined DNA profile data file.

Another aspect of the disclosed embodiments includes a non-transitory computer-readable storage medium, including executable instructions that, when executed by a processor, facilitate performance of operations. The operations include receiving first initial DNA profile data from a first computing device, receiving second initial DNA profile data from a second computing device, and compiling the first initial DNA profile data and second initial DNA profile data into a first combined DNA profile data file.

Another aspect of the disclosed embodiments includes a system for assisting in generating a family tree. The system includes a processor and a memory instructions that, when executed by the processor, cause the processor to receive a plurality of individual DNA profiles; receive relationship assignments or determining relationship assignments relating individual DNA profiles; and, responsive to the relationship assignments and the individual DNA profiles, and then compile a pedigree file.

Another aspect of the disclosed embodiments includes a method for generating a family tree. The method includes receiving a plurality of individual DNA profiles; receiving or relationship assignments or determining relationship assignments relating individual DNA profiles; and responsive to the relationship assignments and the individual DNA profiles, compiling a pedigree file.

Another aspect of the disclosed embodiments includes a non-transitory computer-readable storage medium, including executable instructions that, when executed by a processor, facilitate performance of operations. The operations include receiving a plurality of individual DNA profiles; receiving relationship assignments relating individual DNA profiles; and responsive to the relationship assignments and the individual DNA profiles, compiling a pedigree file.

These and other aspects of the present disclosure are disclosed in the following detailed description of the embodiments, the appended claims, and the accompanying figures.

### BRIEF DESCRIPTION OF THE DRAWINGS

The disclosure is best understood from the following detailed description when read in conjunction with the accompanying drawings. It is emphasized that, according to common practice, the various features of the drawings are not to scale. On the contrary, the dimensions of the various features are arbitrarily expanded or reduced for clarity.

FIG. 1 generally illustrates a computing device in accordance with some aspects of the present disclosure.

FIG. 2 is a flow diagram generally illustrating a method for computer analysis in accordance with some aspects of the present disclosure.

FIG. 3 is a flow diagram generally illustrating a method for generating a family tree in accordance with some aspects of the present disclosure.

FIG. 4 generally illustrates a computer analysis system in accordance with some aspects of the present disclosure.

### DETAILED DESCRIPTION

The following discussion is directed to various embodiments of the invention. Although one or more of these embodiments may be preferred, the embodiments disclosed should not be interpreted, or otherwise used, as limiting the scope of the disclosure, including the claims. In addition, one skilled in the art will understand that the following description has broad application, and the discussion of any embodiment is meant only to be exemplary of that embodiment, and not intended to intimate that the scope of the disclosure, including the claims, is limited to that embodiment.

As described, various laboratory software is used to analyze and present information relating to deoxyribonucleic acid (DNA) identification. In some cases, the DNA profiles of individuals from different sources may differ and need to be reconciled. Further, in some cases, DNA information may be useful in generating family trees. As such, software for reconciling DNA profile data may be desirable. Further, software for assisting in generating family trees using DNA profile data may be desirable.

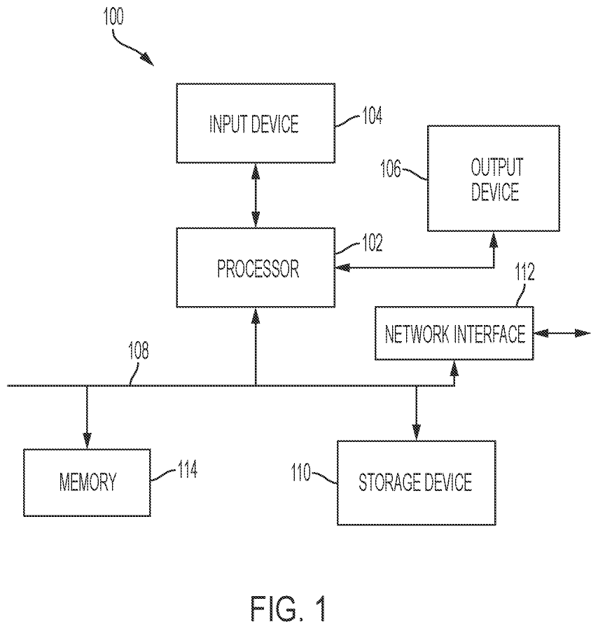

FIG. 1 generally illustrates a computing device 100 according to the principles of the present disclosure. The computing device 100 may be configured to perform various operations and methods. The computing device 100 may include a processor 102 configured to control the overall operation of computing device 100. It should be understood that the processor 102 (e.g., and/or any processors described herein) may include any suitable processor, including those described herein. The computing device 100 may also include a user input device 104 that is configured to receive input from a user of the computing device 100 and to communicate signals representing the input received from the user to the processor 102. For example, the user input device 104 may include a button, keypad, dial, touch screen, audio input interface, visual/image capture input interface, input in the form of sensor data, etc.

The computing device 100 may include an output device 106 (e.g., a display screen, speaker, or any other suitable output device) that may be controlled by the processor 102 to present information to the user. A data bus 108 may be configured to facilitate data transfer between, at least, a storage device 110 and the processor 102. The computing device 100 may also include a network interface 112 configured to couple or connect the computing device 100 to various other computing devices or network devices via a network connection, such as a wired or wireless connection. In some embodiments, the network interface 112 includes a wireless transceiver.

The storage device 110 may include a single disk or a plurality of disks (e.g., hard drives), one or more solid-state drives, one or more hybrid hard drives, and the like. The storage device 110 may include a storage management module that manages one or more partitions within the storage device 110. The computing device 100 may also include a memory 114. The memory 114 may include Random Access Memory (RAM), a Read-Only Memory (ROM), or a combination thereof. In some embodiments, either or both of the storage device 110 and the memory 114 may include flash memory, semiconductor (solid-state) memory, or the like. Either or both of the storage device 110 and the memory 114 may include Random Access Memory (RAM), a Read-Only Memory (ROM), or a combination thereof. The memory 114 may store programs, utilities, or processes to be executed by the processor 102. The memory 114 may provide volatile data storage, and stores instructions related to the operation of the computing device 100.

In some embodiments, the computing device 100 may employ more components or fewer components than those shown in FIG. 1.

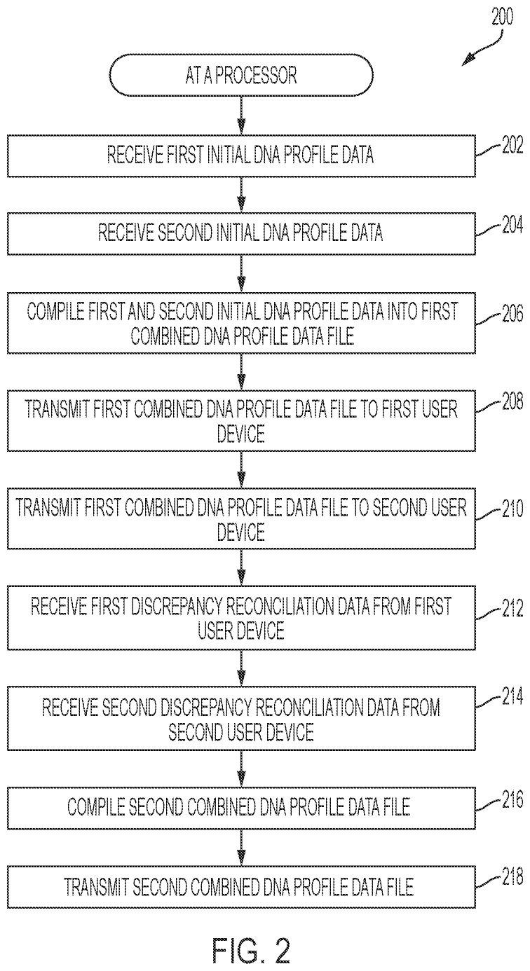

As shown in FIG. 2, a method 200 for laboratory analysis is disclosed. The method 200 may be implemented on one or more computing devices, such as the computing device 100. In some embodiments, the steps of the method 200 may be stored as instructions on a non-transitory computer-readable storage medium (e.g., storage device 110), where the instructions, when executed by a processor (e.g., processor 102), cause the processor to perform the steps of the method 200. In some embodiments, the method 200 may be implemented on a system including a processor (e.g., the processor 102) and a memory (e.g., memory 114) or storage device (e.g., storage device 110), where the memory or storage device includes instructions that, when executed by the processor, cause the processor to perform the steps of the method 200.

In some embodiments, at 202, the method 200 receives a first initial DNA profile data from a first computing device. In some embodiments, the first computing device may be a computing device (e.g., such as a computer workstation or other suitable computing device). The computing device may include features similar to those of the computing device 100 and may be in communication with a network. In some embodiments, the first computing device may have received the first initial DNA profile data from another device (e.g., by way of the network interface 112), have had the first initial DNA profile data entered by a user (e.g., by way of the input device 104), or generate the first initial DNA profile data based on other data received by or entered into the first computing device.

In some embodiments, at 204, the method 200 receives initial DNA profile data from a second computing device. In some embodiments, the second computing device may be a computing device, such as the computing device 100, in connection with the network. In some embodiments, the second computing device may have received the second initial DNA profile data from another device (e.g., by way of the network interface 112), have had the second initial DNA profile data entered by a user (e.g., by way of the input device 104), or generated the second initial DNA profile data based on other data received by or entered into the second computing device.

In some embodiments, the method 200 may include receiving initial DNA profile data from more than two computing devices. It should be understood that the method 200 may receive data from any suitable number of computing devices and any suitable computing devices in addition to or instead of those described herein.

In some embodiments, at 206, the method 200 compiles the first initial DNA profile data and second initial DNA profile data into a first combined DNA profile data file including differences between the first initial DNA profile data and second initial DNA profile data.

In some embodiments, at 208, the method 200 transmits the first combined DNA profile data file to a first user device. In some embodiments, the first user device may be a computing device, such as the computing device 100, in connection with the network. In some embodiments, the first user device may be the same device as the first computing device. In some embodiments, the first user device may be a different device from the first computing device.

In some embodiments, at 210, the method 200 transmits the first combined DNA profile data file to a second user device. In some embodiments, the second user device may be a computing device, such as the computing device 100, in connection with the network. In some embodiments, the second user device may be the same device as the second computing device. In some embodiments, the second user device may be a different device from the second computing device.

In some embodiments, at 212, the method 200 receives first discrepancy reconciliation data from the first user device. In some embodiments, the first discrepancy reconciliation data may be data entered by a user of the first user device (e.g., by way of an input device, such as the input device 104) or determined by the first user device (e.g., by way of a processor, such as the processor 102) reconciling discrepancies between the first initial DNA profile data and second initial DNA profile data.

In some embodiments, at 214, the method 200 receives second discrepancy reconciliation data from the second user device. In some embodiments, the second discrepancy reconciliation data may be data entered by a user of the second user device (e.g., by way of an input device, such as the input device 104) or determined by the second user device (e.g., by way of a processor, such as the processor 102) reconciling discrepancies between the first initial DNA profile data and second initial DNA profile data. In some embodiments, the method 200 may include analyzing highlighted profiles that are reconciled. The method 200 may further include determining how to reconcile the highlighted profiles.

At 216, the method 200 may include, responsive to the first discrepancy reconciliation data and the first discrepancy reconciliation data, compiling the first initial DNA profile data and second initial DNA profile data into a second combined DNA profile data file. This may allow for a reviewed and verified second combined DNA profile data file.

In some embodiments, at 218, the method 200 may include transmitting the second combined DNA profile data. In some embodiments, the second combined DNA profile data may be transmitted to another computing device (e.g., a server, remote database, etc.), such as the computing device 100. From there, the DNA profile data may be presented by way of an output device, such as the output device 106.

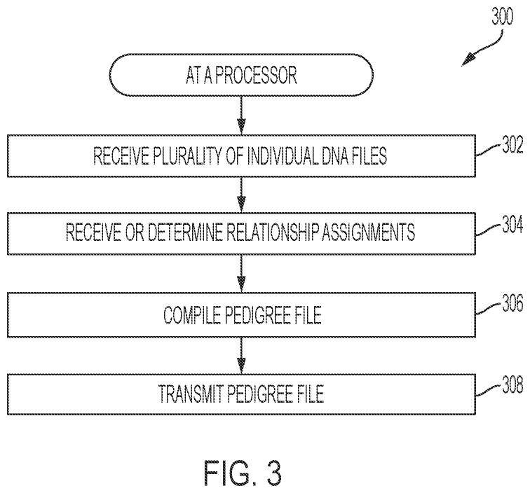

As shown in FIG. 3, a method 300 for generating a family tree is disclosed. The method 300 may be implemented on one or more computing devices, such as the computing device 100. In some embodiments, the steps of the method 300 may be stored as instructions on a non-transitory computer-readable storage medium (e.g., storage device 110), where the instructions, when executed by a processor (e.g., processor 102), cause the processor to perform the steps of the method 300. In some embodiments, the method 300 may be implemented on a system including a processor (e.g., the processor 102) and a memory (e.g., memory 114) or storage device (e.g., storage device 110), where the memory or storage device includes instructions that, when executed by the processor, cause the processor to perform the steps of the method 300.

In some embodiments, at 302, the method 300 may include receiving a plurality of individual DNA profiles. The individual DNA profiles may be known to be related or postulated to be related.

In some embodiments, at 304, the method 300 may include receiving relationship assignments or determining relationship assignments relating to individual DNA profiles. The relationship assignments include information relating to the individuals in some relationships (e.g., parent-child relationship, sibling relationship, etc.).

In some embodiments, at 306, the method 300 may include, responsive to the relationship assignments and the individual DNA profiles, compiling a pedigree file. The pedigree file contains the individuals known and the relationships between the individuals. The pedigree file may be configured to display as a family tree. In some embodiments, the individuals and relationships in a pedigree or family tree may be represented graphically as a circle for female, a square for male, a diamond for someone of unknown gender, a horizontal line for a spousal relationship, a vertical line for a parent-child relationship, and a bracket shaped line for a sibling relationship. These examples are non-limiting, and other suitable configurations are contemplated.

In some embodiments, at 308, the method 300 may include transmitting the pedigree file. The file may be presented (e.g., by way of an output device, such as output device 106), or stored in a database (e.g., by way of a memory, such as memory 114).

It should be noted that the method 200 and the method 300 can each include additional or fewer steps, and the steps may occur in any suitable order.

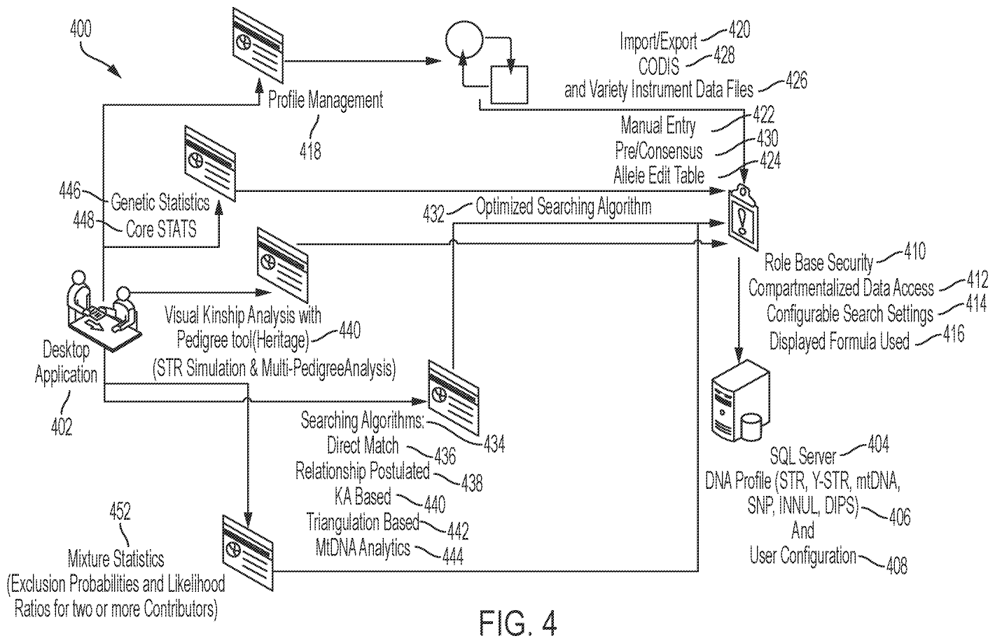

As shown in FIG. 4, an exemplary system 400 may include a software suite that may be used to assist users (e.g., scientists or other suitable users) in the DNA Identification process through the use of statistics and searching algorithms. In some embodiments, the system 400 may be implemented as a desktop application 402. In some embodiments, the system 400 provides a flexible, comprehensive set of tools to satisfy the functional needs of various organizations while optimizing the ease of use for users. In some embodiments, the system 400 may be valuable for combinatorial evaluation of available genetic information.

In some embodiments, the system 400 may perform functions including a combination of one or more of Role-Based User Management, Case Set Management, Numbering System Configuration, Statistical Configuration, Searching Configuration, Profile Management, STR (short tandem repeat) and SNPs (single nucleotide polymorphisms) Allele Frequency Database Management, mitochondrial DNA (mtDNA) and Y-STR (STR on the Y-chromosome) Database Management, Multiplex Definition Management, Population Group Management, Loci Management, Mito Reference Sequence Management, Genotype Frequency Statistics, Kinship Analysis (including STR Simulations and Multi-Pedigree Analysis), Direct Match Searching, Postulated Relationship Likelihood Ratio Searching, Pedigree-UHR (Unidentified Human Remains) Filter-Based Kinship Analysis Searching, Pedigree-UHR Triangulation-Based Searching, mtDNA-Specific Analysis, Exclusion Probability calculations for STR Mixtures, and Likelihood Ratio calculations for STR Mixtures, and any other suitable functionality.

In some embodiments, the system 400 may store persistent data in a database 404, which may include a structured query language (SQL) Server database or any other suitable database. In some embodiments, the database 404 may be chosen for flexibility and ease of management. In some embodiments, the database architecture may allow the system 400 to store different DNA data type and DNA Profile 406 information such as STR information, mitochondrial information, SNP information, Y-STR information, and other suitable types of information. In some embodiments, the database 404 may store user configuration data 408, including user roles profile information, and archival profile data.

In some embodiments, the system 400 may include role-based security 410 based on a user's application interface (UI) role(s) and/or user's data access role (e.g., compartmentalized data access 412). In some embodiments, the material to access may be defined as case sets or a set of profiles. In some embodiments, authentication functionality may use various authentication techniques (e.g., using a user name and password or other suitable information), certificate authentication using a common access card (CAC), or any other suitable authentication mechanism or process. In some embodiments, the system 400 may include role-based access for user interface (UI), a security feature that may be designed to grant user access to different UIs and/or functions within the system. In some embodiments, a user accessing the same screen may not be able to see and/or able to access all available features on that screen, depending on their data access role(s).

In some embodiments, the system 400 may include compartmentalized data access 412, a security feature that may be designed to allow a user to only see the data that they have access to. For example, a user assigned to just case sets A and B will only see case sets A and B and not case set C.

In some embodiments, the system 400 may include configurable search settings 414, with which the system manages, saves, and reuses particular templates, which may contain dozens of statistical, search, and kinship analysis related settings used throughout the system. In some embodiments, different analytical configurations can be created for a different set of users or specific standard operating procedures. In some embodiments, the system 400 may display the formula 416 used (e.g., on an output device, such as output device 106) for the different analytical configurations. In some embodiments, the system 400 may allow for these settings to be ‘overridden’ on individual screens without revising the actual ‘template’ while using most of the functionality within the system.

In some embodiments, the DNA profiles may be stored separately by multiplex definition (DNA Kit). In some embodiments, the DNA profiles (based on profile name) may be combined to create a composite profile called the profile suite. In some embodiments, the system 400 may store and track STR, Y-STR, X-STR, mtDNA, SNP, insertion and null alleles (INNUL), and effective interfering particles (DIPS) DNA profiles.

In some embodiments, the system 400 may include profile management 418 functionality, in which the main DNA profile data management allows a user to input and/or import and export 420 DNA profile data in different formats.

In some embodiments, the system 400 may include manual entry 422 functionality. With manual entry 422, the user can manually enter DNA profiles such as allele data for STR, Y-STR and X-STR and regions/polymorphisms for mtDNA. In some embodiments, this data may be stored in an editable allele edit table 424.

In some embodiments, the system 400 may include import functionality, in which, instead of manually entering DNA profile data into the system, the user can import data directly from a text file that is produced by a variety of instruments (i.e., a variety instrument data file 426). Non-limiting examples of the text files may include many various formats such as combined DNA index system (CODIS) 428 format, tab delimited format, GeneMapper, and Genotyper.

In some embodiments, the system 400 may include a pre-consensus and consensus 430 process, an alternative way to import data into the system. In some embodiments, the pre-consensus and consensus 430 process may allow multiple users to review the DNA profile data before it becomes available in the searchable DNA profile database e.g., stored on the database 404). In some embodiments, the pre-consensus and consensus process 430 may receive multiple DNA profiles created by different users, provide a composite DNA profile, and allow users to resolve discrepancies.

In some embodiments, the system 400 includes export functionality. In some embodiments, the system 300 using or performing the export functionality, exports DNA profile data from the system 400 and outputs an interchangeable text file format to be used by other systems. Examples of formats are CODIS 428, tab delimited format, various proprietary formats, or any other suitable formats.

In some embodiments, the system 400 may provide random STR profile generation functionality, in which the system 400 may generate STR profiles based on frequency data set up in the system. In some embodiments, random STR profile generation functionality may be useful in the study of profile variability if real-world data is unavailable or insufficient.

In some embodiments, the system 400 allows the user to enter and configure new statistical analysis data according to a lab's specific needs. In some embodiments, the system 400 may assist users in the DNA identification process through the use of a variety of statistics and searching algorithms. In some embodiments, the searching algorithms may include optimized searching algorithms 432. In some embodiments, the system 400 may perform genetic searching and statistical analyses as described herein.

In some embodiments, the system 400 may include various search algorithms 434 for a search engine. In some embodiments, the search engine may be configured to provide different kinds of search types, from direct match 436 to relationship postulated 438, kinship analysis based 440, triangulation based 442, and mtDNA Analytics 444.

In some embodiments, the system 400 may include combined searches for multiple types of DNA profiles. In some embodiments, the combined searching may include the ability to run one single search operation and combine the statistical results from STR, mtDNA, Y-STR, X-STR, and SNPs.

In some embodiments, the system 400 may include STR direct match search functionality. In some embodiments, basic types of DNA searching are based around the interest in matching DNA profiles. This refers to the comparison of individual allele values for two profiles in a one-to-many or many-to-many search. In some embodiments, the system 400 performs the comparison via basic logic and text comparisons as opposed to other types of searching involving genetic statistics 446 and/or CORE statistics 448. In some embodiments, multiple options for reporting are available. In some embodiments, the system 400 may save search results, analytical configuration settings, and numerous types of reports for later review.

In some embodiments, the system 400 may include functionality for postulating relationships. In some embodiments, the system 400 may include ‘canned’ relationship scenarios such as parent/child and siblings. This may follow the same basic paradigm as direct match searching, where one-to-many or many-to-many searches for different types of profiles suites originating from Case Sets or hand-selected data sets may be performed. Unlike direct match searching, relationship postulated searching may perform a statistical analysis calculation for each profile suite comparison (and for each population group) during the search. Likelihood ratios may be provided to show how likely the relationship is for each search result.

In some embodiments, the system 400 may include kinship analysis based searching. In some embodiments, kinship analysis based searching may include the ability to screen unidentified human remains against the ‘target’ designated in the pedigrees of a particular case set of profiles interest, substituting that particular ‘target’ with a single or all incident unidentified human remains. In some embodiments, there is no need to go into the kinship analysis module proper and substitute the profile for the ‘target’ there.

In some embodiments, the system 400 may include triangulation based searching. Triangulation based searching may include heuristics based on parentage, siblingship, and direct match. In some embodiments, the algorithm is specialized for mass disaster situations. In some embodiments, the search may involve comparing pedigrees against UHRs and performing pair wise comparisons among the UHR and individual family members instead of full Kinship Analysis. In some embodiments, triangulation searching may provide clues as to where to further look for matches. In some embodiments, triangulation-based searching may provide convenience for DNA forensics searching.

In some embodiments, the system 400 may include mtDNA analytics. In some embodiments, the mtDNA specific searching tools may use ‘polymorphism’ based search rules as well as an option to use a different but known, instead, to be more a correct comparison ‘string’ search. In some embodiments, the mtDNA Analytics may be similar to direct match searching but restricted to mtDNA.

In some embodiments, the system 400 may include general statistics functionality, including the ability to select profiles of a known direct match or relationship postulated situation to immediately return and report on just the statistics. These may be used in situations where there is no need to run a search.

In some embodiments, the system 400 may include kinship analysis functionality, such as a pedigree drawing tool, STR simulation, and pedigree multiple profile runs.

In some embodiments, the system 400 may include kinship analysis. Kinship analysis may determine the relationship validity of user-defined pedigree and reports likelihood ratios based on user selected ‘target’ person(s) on the pedigree of interest. In some embodiments, kinship analysis may be used to calculate likelihood ratios for different populations groups and return a cumulative likelihood when the profile has been typed for different DNA profile types including STR, mtDNA, Y-STR, and SNPs.

In some embodiments, the system 400 may include STR simulation, also known as ‘Monte Carlo’ simulation. This is the random generation (e.g., by a computer algorithm) of DNA profiles for specific persons in a pedigree that have no DNA available. In some embodiments, profiles are randomly assigned, and then the range of would-be likelihood ratios is provided to discern the relative value of obtaining a DNA profile for a particular person with no DNA available and calculates a percentile simulation distribution of the likelihood ratios.

In some embodiments, the system 400 may include functionality for performing multiple pedigree profile runs. In some embodiments, this functionality may provide the ability to assign multiple profiles to the designated ‘target’ person to avoid having to do individual runs for multiple profiles. In some embodiments, this may allow for cherry-picking or use with unidentified human remains.

In some embodiments, the system 400 includes a heritage pedigree tool 450. In some embodiments, the heritage pedigree tool may be a visual pedigree tool that allows users to draw family trees and to assign DNA profiles that can be used with kinship analysis. In some embodiments, after the profile is assigned to the relationship in the pedigree tool, it displays the profile name and color codes sections in the relationship on the pedigree, which designates the type of profile including STR, mtDNA, Y-STR, and SNPs. In some embodiments, the pedigree has the option to print the pedigree and can be saved in XML format. In some embodiments, the XML formatted file can then be used to load the pedigree into another scenario or on another installation.

In some embodiments of the system 400, metadata may be saved in XML format for flexibility and ease of integration with other systems. In some embodiments, this information may be stored in the database 404 to be retrieved later to redraw the graphic on the display. In some embodiments, the interface between user and program may be through the keyboard and mouse.

In some embodiments, the individuals and relationships in a pedigree may be represented graphically as a circle for female, a square for male, a diamond for someone of an unknown gender, a horizontal line for a spousal relationship, a vertical line for a parent-child relationship, and a bracket shaped line for a sibling relationship. These examples are non-limiting, and other suitable configurations are contemplated.

In some embodiments, popup menu items may appear upon the user performing a right-click on the pedigree itself. Examples include: add male, add female, add unknown, new pedigree, open pedigree, save pedigree, view/edit XML, save as image, print pedigree, zoom in/out/reset, and select all (pedigree individuals). These examples are non-limiting, and other suitable configurations are contemplated.

In some embodiments, popup menu items may appear upon the user performing a right-click on an individual in the pedigree. Examples include: add male relative {father, brother, son}, add female relative {mother, sister, daughter}, add spouse, add monozygous twin, add dizygous twin, change gender {swap gender, to male, to female, to unknown, to other}, toggle vital status, toggle divorced status, make target, simulate STR, delete profile suite, consanguineous. These examples are non-limiting, and other suitable configurations are contemplated.

In some embodiments, the system 400 may include mixture statistics functionality 452, functionality that analyzes DNA profile mixtures such as exclusion probabilities for particular mixture profiles, as well as likelihood ratios for two or more contributors. A DNA mixture profile is defined by the presence of three or more alleles at one or more typed loci.

In some embodiments, a system for analysis is disclosed. The system includes a processor; and a memory including instructions that, when executed by the processor, cause the processor to: receive first initial DNA profile data from a first computing device; receive second initial DNA profile data from a second computing device; and compile the first initial DNA profile data and second initial DNA profile data into a first combined DNA profile data file.

In some embodiments, the instructions further cause the processor to transmit the first combined DNA profile data file to a first user device and transmit the first combined DNA profile data file to a second user device (not e.g., such as a computer workstation or other suitable computing device). In some embodiments, the instructions further cause the processor to receive first discrepancy reconciliation data from the first user device and receive second discrepancy reconciliation data from the second user device. In some embodiments, the instructions further cause the processor to, responsive to the first discrepancy reconciliation data and the first discrepancy reconciliation data, compile the first initial DNA profile data and second initial DNA profile data into a second combined DNA profile data file. In some embodiments, the instructions (e.g., which may be initiated by the user) further cause the processor to transmit the second combined DNA profile data.

In some embodiments, a method for analysis is disclosed. The method includes receiving first initial DNA profile data from a first computing device, receiving second initial DNA profile data from a second computing device, and compiling the first initial DNA profile data and second initial DNA profile data into a first combined DNA profile data file. In some embodiments, the method further includes transmitting the first combined DNA profile data file to a first user device and transmitting the first combined DNA profile data file to a second user device. In some embodiments, the method further includes receiving first discrepancy reconciliation data from the first user device and receiving second discrepancy reconciliation data from the second user device. In some embodiments, the method further includes, responsive to the first discrepancy reconciliation data and the first discrepancy reconciliation data, compiling the first initial DNA profile data and second initial DNA profile data into a second combined DNA profile data file. In some embodiments, the method includes transmitting the second combined DNA profile data.

In some embodiments, a non-transitory computer-readable storage medium is disclosed. The non-transitory computer-readable storage medium includes executable instructions that, when executed by a processor, facilitate performance of operations, including receiving first initial DNA profile data from a first computing device, receiving second initial DNA profile data from a second computing device, and compiling the first initial DNA profile data and second initial DNA profile data into a first combined DNA profile data file. In some embodiments, the instructions further include transmitting the first combined DNA profile data file to a first user device and transmitting the first combined DNA profile data file to a second user device. In some embodiments, the instructions further include receiving first discrepancy reconciliation data from the first user device and receiving second discrepancy reconciliation data from the second user device. In some embodiments, the instructions further include, responsive to the first discrepancy reconciliation data and the first discrepancy reconciliation data, compiling the first initial DNA profile data and second initial DNA profile data into a second combined DNA profile data file. In some embodiments, the instructions further include transmitting the second combined DNA profile data.

In some embodiments, a system for graphically presenting/generating a family tree is disclosed. The system includes a processor and a memory including instructions that, when executed by the processor, cause the processor to receive a plurality of individual DNA profiles; receive relationship assignments or determining relationship assignments relating individual DNA profiles; and responsive to the relationship assignments and the individual DNA profiles, compile a pedigree file. In some embodiments, the instructions further cause the processor to transmit the pedigree file as an output file.

In some embodiments, a method for generating a family tree is disclosed. The method includes receiving a plurality of individual DNA profiles; receiving or relationship assignments or determining relationship assignments relating individual DNA profiles; and responsive to the relationship assignments and the individual DNA profiles, compiling a new pedigree file. In some embodiments, the method further includes transmitting the pedigree file.

A non-transitory computer-readable storage medium, including executable instructions that, when executed by a processor, facilitate performance of operations, including receiving a plurality of individual DNA profiles; receiving relationship assignments relating individual DNA profiles; and, responsive to the relationship assignments and the individual DNA profiles, compiling a pedigree file. In some embodiments, the instructions further include transmitting the pedigree file.

In some embodiments, a system for analysis of a graphically presented family tree is disclosed. The system comprises a processor, and a memory including instructions that, when executed by the processor, cause the processor to receive first initial deoxyribonucleic acid (DNA) profile data corresponding to a first person, from a first computing device; receive second initial DNA profile data corresponding to a second person, from a second computing device; receive relationship data defining the relationship between the first person and the second person; display a visualization of the relationship between the first and second person; receive a request to search a database of DNA profiles, the search request comprising at least one statistical model, at least one search algorithm, and mixture statistics based on the DNA profiles of the first and second person; receive from the database of DNA profiles the closest matching DNA profiles; and display to the user the results of the search.

In some embodiments, a system for analysis of a graphically presented family tree is disclosed. The system further comprises receiving the incomplete DNA profile of a third person, the system causes the processor to receive from the user a request to randomly generate values to fill the DNA profile, in view of the likelihood ratios generated based on the first and second person's DNA profiles.

In some embodiments, a system for analysis of a graphically presented family tree is disclosed. The system further comprises altering the color of the representation of an individual in the visualization based on which type of DNA profile is associated with that individual.

In some embodiments, a system for analysis of a graphically presented family tree is disclosed. The system further comprises receiving a second DNA profile for the first person; determining a discrepancy between the two DNA profiles associated with the first person; generating a combined DNA profile and sending it the first person; and receiving, from the first person, discrepancy reconciliation data and fixing the discrepancies in the data profile based on the discrepancy reconciliation data.

In some embodiments, a system for analysis of a graphically presented family tree is disclosed. The system further comprises requiring authentication of the user before granting access to the visualization display and any of its associated functionality.

The above discussion is meant to be illustrative of the principles and various embodiments of the present disclosure. Numerous variations and modifications will become apparent to those skilled in the art once the above disclosure is fully appreciated.

The word “example” is used herein to mean serving as an example, instance, or illustration. Any aspect or design described herein as “example” is not necessarily to be construed as preferred or advantageous over other aspects or designs. Rather, use of the word “example” is intended to present concepts in a concrete fashion. As used in this application, the term “or” is intended to mean an inclusive “or” rather than an exclusive “or.” That is, unless specified otherwise, or clear from context, “X includes A or B” is intended to mean any of the natural inclusive permutations. That is, if X includes A; X includes B; or X includes both A and B, then “X includes A or B” is satisfied under any of the foregoing instances. In addition, the articles “a” and “an” as used in this application should generally be construed to mean “one or more” unless specified otherwise or clear from context to be directed to a singular form. Moreover, use of the term “an implementation” or “one implementation” throughout is not intended to mean the same embodiment or implementation unless described as such.

Implementations the systems, algorithms, methods, instructions, etc., described herein can be realized in hardware, software, or any combination thereof. The hardware can include, for example, computers, intellectual property (IP) cores, application-specific integrated circuits (ASICs), programmable logic arrays, optical processors, programmable logic controllers, microcode, microcontrollers, servers, microprocessors, digital signal processors, or any other suitable circuit. The term “processor” should be understood as encompassing any of the foregoing hardware, either singly or in combination. The terms “signal” and “data” are used interchangeably.

As used herein, the term module can include a packaged functional hardware unit designed for use with other components, a set of instructions executable by a controller (e.g., a processor executing software or firmware), processing circuitry configured to perform a particular function, and a self-contained hardware or software component that interfaces with a larger system. For example, a module can include an application specific integrated circuit (ASIC), a Field Programmable Gate Array (FPGA), a circuit, digital logic circuit, an analog circuit, a combination of discrete circuits, gates, and other types of hardware or combination thereof. In other embodiments, a module can include memory that stores instructions executable by a controller to implement a feature of the module.

Further, in one aspect, for example, systems described herein can be implemented using a general-purpose computer or general-purpose processor with a computer program that, when executed, carries out any of the respective methods, algorithms, and/or instructions described herein. In addition, or alternatively, for example, a special purpose computer/processor can be utilized which can contain other hardware for carrying out any of the methods, algorithms, or instructions described herein.

Further, all or a portion of implementations of the present disclosure can take the form of a computer program product accessible from, for example, a computer-usable or computer-readable medium. A computer-usable or computer-readable medium can be any device that can, for example, tangibly contain, store, communicate, or transport the program for use by or in connection with any processor. The medium can be, for example, an electronic, magnetic, optical, electromagnetic, or a semiconductor device. Other suitable mediums are also available.

What is claimed is:

1. A system for analysis, the system comprising:
a processor; and
a memory including instructions that, when executed by the processor, cause the processor to:
receive first initial deoxyribonucleic acid (DNA) profile data from a first computing device;
receive second initial DNA profile data from a second computing device; and
compile the first initial DNA profile data and second initial DNA profile data into a first combined DNA profile data file.

2. The system of claim 1, wherein the instructions further cause the processor to:
transmit the first combined DNA profile data file to a first user device; and
transmit the first combined DNA profile data file to a second user device.

3. The system of claim 2, wherein the instructions further cause the processor to:
receive first discrepancy reconciliation data from the first user device; and
receive second discrepancy reconciliation data from the second user device.

4. The system of claim 3, wherein the instructions further cause the processor to, responsive to the first discrepancy reconciliation data and the first discrepancy reconciliation data, compile the first initial DNA profile data and second initial DNA profile data into a second combined DNA profile data file.

5. The system of claim 4, wherein the instructions further cause the processor to transmit the second combined DNA profile data.

6. A method for analysis, the method comprising:
receiving first initial deoxyribonucleic acid (DNA) profile data from a first computing device;
receiving second initial DNA profile data from a second computing device; and
compiling the first initial DNA profile data and second initial DNA profile data into a first combined DNA profile data file.

7. The method of claim 6, further comprising:
transmitting the first combined DNA profile data file to a first user device; and
transmitting the first combined DNA profile data file to a second user device.

8. The method of claim 7, further comprising:
receiving first discrepancy reconciliation data from the first user device; and
receiving second discrepancy reconciliation data from the second user device.

9. The method of claim 8, further comprising, responsive to the first discrepancy reconciliation data and the first discrepancy reconciliation data, compiling the first initial DNA profile data and second initial DNA profile data into a second combined DNA profile data file.

10. The method of claim 9, further comprising transmitting the second combined DNA profile data.

11. A non-transitory computer-readable storage medium, comprising executable instructions that, when executed by a processor, facilitate performance of operations, comprising:
receiving first initial deoxyribonucleic acid (DNA) profile data from a first computing device;
receiving second initial DNA profile data from a second computing device; and
compiling the first initial DNA profile data and second initial DNA profile data into a first combined DNA profile data file.

12. The non-transitory computer-readable storage medium of claim 11, wherein the instructions further comprise:
transmitting the first combined DNA profile data file to a first user device; and
transmitting the first combined DNA profile data file to a second user device.

13. The non-transitory computer-readable storage medium of claim 12, wherein the instructions further comprise:
receiving first discrepancy reconciliation data from the first user device; and
receiving second discrepancy reconciliation data from the second user device.

14. The non-transitory computer-readable storage medium of claim 13, wherein the instructions further comprise, responsive to the first discrepancy reconciliation data and the first discrepancy reconciliation data, compiling the first initial DNA profile data and second initial DNA profile data into a second combined DNA profile data file.

15. The non-transitory computer-readable storage medium of claim 14, wherein the instructions further comprise transmitting the second combined DNA profile data.

16. A system for generating a family tree, the system comprising:
a processor; and
a memory including instructions that, when executed by the processor, cause the processor to:
receive a plurality of individual deoxyribonucleic acid (DNA) profiles;
receive relationship assignments or determining relationship assignments relating individual DNA profiles; and
responsive to the relationship assignments and the individual DNA profiles, compile a pedigree file.

17. The system of claim 16, wherein the instructions further cause the processor to transmit the pedigree file.

18. A method for generating a family tree, the method comprising:
receiving a plurality of individual deoxyribonucleic acid (DNA) profiles;
receiving or relationship assignments or determining relationship assignments relating individual DNA profiles; and
responsive to the relationship assignments and the individual DNA profiles, compiling a pedigree file.

19. The method of claim 18, the method further comprising transmitting the pedigree file.

20. A non-transitory computer-readable storage medium, comprising executable instructions that, when executed by a processor, facilitate performance of operations, comprising:
receiving a plurality of individual deoxyribonucleic acid (DNA) profiles;
receiving relationship assignments relating individual DNA profiles; and
responsive to the relationship assignments and the individual DNA profiles, compiling a new pedigree file.
